(19) United States
(12) Patent Application Publication
(10) Pub. No.: US 2020/0098449 A1
(43) Pub. Date: Mar. 26, 2020

(54) REMOTE-CONTROLLED AUTOMATED SYSTEM FOR DRUG TESTING AND SCREENING

(71) Applicant: Phylumtech S.A., Sunchales (AR)

(72) Inventors: Sergio Hernan SIMONETTA, Ataliva (AR); Mariano Javier SANTA CRUZ, Sunchales (AR)

(21) Appl. No.: 16/576,041

(22) Filed: Sep. 19, 2019

Related U.S. Application Data
(60) Provisional application No. 62/733,653, filed on Sep. 20, 2018.

(51) Int. Cl. G16C 20/30; G16C 20/80; G16C 20/70; G16H 10/40; G06N 20/00; G16H 70/40

(52) U.S. Cl. CPC G16C 20/30; G16C 20/80; G16H 70/40; G16H 10/40; G06N 20/00; G16C 20/70

(57) ABSTRACT

A remote-controlled automated system for drug testing and screening. Systems and methods for the discovery of new pharmaceuticals according to their toxicity and/or efficacy, where the discovery process is guided by a computer assisted system and performed into a remote laboratory; additionally, a machine learning algorithm is configured to obtain the results.

### FIELD OF THE INVENTION

The invention relates to a remote-controlled automated system for drug testing and screening. More particularly, the invention provides systems and methods for the discovery of new pharmaceuticals according to their toxicity and/or efficacy, where the discovery process is guided by a computer assisted system and performed into a remote laboratory; additionally, a machine learning algorithm is configured to obtain the results of the experimental results and use these results' data to recommend experimental parameters in a future experiment.

### BACKGROUND OF THE INVENTION

The identification of new drug candidates, and the process of transforming these into high-content lead series, are key activities in modern drug discovery. The decisions taken during this process have far-reaching consequences for success later in lead optimization and even more crucially in clinical development. Recently, there has been an increased focus on these activities due to escalating downstream costs resulting from high clinical failure rates. In addition, the vast emerging opportunities from efforts in functional genomics and proteomics demands a departure from the linear process of identification, evaluation and refinement activities towards a more integrated parallel process. This calls for flexible, fast and cost-effective strategies to meet the demands of producing high-content lead series with improved prospects for clinical success. see Bleicher, K. H., Böhm, H. J., Müller, K., &. Alanine, A. I. (2003). A guide to drug discovery: hit and lead generation: beyond high-throughput screening. Nature reviews Drug discovery, 2(5), 369.

Two main bottlenecks in the discovery of new drugs are the high cost of implementation of new projects and the low predictability of results. Screening of new molecular entities against a biological target is a time consuming process, requiring not only the specialized know how but also the proper laboratory infrastructure.

Starting a new project for drug discovery requires multiple multidisciplinary tasks including the setting of biological experimental paradigms for testing; setting-up of laboratory hardware and software to carry on the experiments, training of lab technicians; selecting and purchasing reactives from different providers, running the actual “wet” experiments and finally acquiring and processing many gigabytes of information to get the results. This laborious work requires multidisciplinary knowledge from specialists in chemistry, bioengineering, biotechnology and bioinformatics, adding a logistic coordination of chemical, biological reactives and equipment provision. The process itself needs several months to be completed; as well as a high cost, which may not be affordable for every lab.

Some solutions to different stages of this problem has been proposed in the past.

The late-stage attrition of chemical entities in development and beyond is highly costly, and therefore such failures must be kept to a minimum by setting in place a rigorous, objective quality assessment at key points in the discovery process. This assessment needs to begin as early as possible and must be of high stringency to prevent precious resources being squandered on less promising lead series and projects. The earliest point at which such knowledge-driven decisions can be made is in the lead-generation phase. Here, the initial actives, or ‘hits’, are progressed into lead series by a comprehensive assessment of chemical integrity, synthetic accessibility, functional behavior, structure-activity-relationships (SAR), as well as bio-physicochemical and absorption, distribution, metabolism and excretion (ADME) properties. This early awareness of the required profile (a given selectivity, solubility, permeation, metabolic stability and so on) is important for the selection and prioritization of series with the best development potential

Rauwerda et al (2006) discloses means to boosting the drug discovery process, specifically dealing with the volume and diversity of data generated. Rauwerda et al (2006) further discloses an enhanced-science (e-science) approach based on remote collaboration, reuse of data and methods, and supported by a virtual laboratory environment promises to get the drug discovery process afloat. Rauwerda at al. (2006) focuses on the creation, use and preservation of information in formalized knowledge spaces is essential to the e-science approach. (see Rauwerda, H., Roos, M., Hertzberger, B. O., & Breit, T. M. (2006). The promise of a virtual lab in drug discovery. Drug discovery today, 11(5-6), 228-236) Rauwerda et al (2006) discloses means for optimizing decisions regarding drug discovery, based on collected data regarding these drugs. However, Rauwerda at al. (2006) does not disclose a system to assess and select drugs by their toxicity and efficacy using in vitro, ex vivo or in vivo systems.

Pitzer, B et al (2012) discloses a remote lab system that allows remote groups to access a shared PR2. This lab enables groups of researchers to participate directly in state-of-the-art robotics research and improves the reproducibility and comparability of robotics experiments. D2 presents solutions to interface, control and design difficulties in the client and server-side software when implementing a remote laboratory architecture (see Pitzer, B., Osentoski, S., Jay, G., Crick, C., & Jenkins, 0. C. (2012, May). Pr2 remote lab: An environment for remote development and experimentation. In Robotics and Automation (ICRA), 2012 IEEE International Conference on (pp. 3200-3205). IEEE). Pitzer, B et al (2012) does not disclose a system to assess and select drugs by their toxicity and efficacy using in vitro, ex vivo or in vivo systems.

US20090247417A1 discloses method and system for drug screening from candidate compounds selected from a library. The system includes multiple hardware components and a computer software system for scheduling and coordinating the operations of the hardware components. The drug screening system is mainly for orchestrating laboratory functions, which automatically assesses samples. However, the system described in US20090247417A1 does not select the relevant experimental protocol. Furthermore, US20090247417A1 does not disclose a system to assess and select drugs by their toxicity and efficacy as well as performing experiments using in vitro, ex vivo or in vivo systems.

WO2004038602A1 discloses integrated spectral data processing, data mining, and modeling system for use in diverse screening and biomarker discovery applications. The system described in WO2004038602A1 provides automated processing of raw spectral data, data standardization, reduction to data to modeling form), and unsupervised and supervised model building, visualization, analysis and prediction. The system incorporates data visualization tools and enables the user to perform visual data mining, statistical analysis and features extraction. WO2004038602A1 discloses a system for drug discovery, using automated processing of raw spectral data However, WO2004038602A1 does not disclose a system to assess and select drugs by their toxicity and efficacy using in vitro, ex vivo or in vivo animal systems.

CA2267769A1 describes an automated drug discovery unit comprising: a) a matrix-with-memory microreactor; b) a compound synthesizer; c) means for sorting the matrix-with-memory microreactors; and d) compound cleavage means for removing compounds from the matrix-with-memory microreactors. CA2267769A1 discloses an automated drug discovery system, including micro reactors. However, CA2267769A1 does not disclose a system to assess and select drugs by their toxicity and efficacy using in vitro, ex vivo or in vivo systems.

However, even these are not integrated solutions, there is a still a problem with the success rate of the results.

The high attrition rate attributed to failures in advanced stages of drug development are the consequence of lack of predictability of animal effects at early stages of discovery.

Some solutions has been already proposed to this problem such as in silico prediction of ADMET properties of compounds. One of the more powerful approaches in these line is the development of the genetic field based on genomic information. Even though non-genetical tools are very useful and encouraging, there are limitations concerning to the lack of experimental homogenization in the comparison of multiple experiments coming from different sources, or the power of information based only in the theoretical correlations.

With the advance of data information processing capability, it will be possible at least in 105 theory to acquire multiple experiments in a standardized way, to estimate new correlations not possible to know in advance and to increase the predictability of the drug passing the DD phase.

Due to the escalating downstream costs in the development phase, objective quality assessment of lead series long before entering clinical trials is an increasing necessity within pharmaceutical research., see Bleicher, K. H., Böhm, H. J., Müller K., & Alanine, A. I. (2003). A guide to drug discovery: hit and lead generation: beyond high-throughput screening. Nature reviews Drug discovery. 2(5), 369.

In this invention we propose an integrated system able to solve these two bottleneck in the drug discovery pipeline. We show an example of implementation of the methodology as a web service.

### SUMMARY OF THE INVENTION

It is thus one object of the present invention to disclose a remote-controlled automated system for drug testing and screening, said system characterized by:
a. a selection web module for providing selected drug discovery experiments, said selection web module comprises a catalog of available experimental protocols, a list of experimental models, and a list of chemical compounds;
b. a remote robotic node configured to run said selected drug discovery experiments and retrieve results of these experiments;
c. a visualization web module configured to analyze and visualize results of said selected drug discovery experiments; and
d. a machine learning algorithm configured to obtain said experimental results and use said results to recommend experimental parameters in a future experiment.

It is another object of the invention to disclose a remote-controlled automated system, wherein said system configured to run a plurality of said drug discovery experiments at the same time period.

It is another object of the invention to disclose a remote-controlled automated system, wherein said system configured to share confidentially said experimental results among users of said system.

It is another object of the invention to disclose a remote-controlled automated system, wherein said users are rewarded for sharing said experimental results among users of said system

It is another object of the invention to disclose a remote-controlled automated system as defined above, wherein said selection web module comprises:
a. a webpage frontend layer; said frontend layer comprises several modules: a protocol catalog, an experimental model selection module), and compound set/treatment module.
b. an administrative layer interconnected with said frontend layer, configured to manage standardized experimental protocols according to parameters set by user of said system.

It is another object of the invention to disclose the administrative layer as defined above, wherein said parameters comprise title, plot, protocol abstract, and any combination thereof.

It is another object of the invention to disclose a remote-controlled automated system as defined above, wherein said remote robotic node comprises:
a. a system backend, said system backend comprising an automatization compilation module); a task scheduler module;
b. an TOT layer, comprising a controller; said controller interconnected to a data base
c. a physical layer comprising at least one device, and laboratory reactives (36); said physical layer is interconnected with lab stock management.

It is another object of the invention to disclose a remote-controlled automated system as defined above, wherein said recording web module comprises:
a. an AI module;
b. a data presenter;
c. a lab stock management; and
d. a visualization layer.

It is another object of the invention to disclose a remote-controlled automated system as defined above, wherein said system additionally comprises a blockchain module configured to transaction of secured information available from regulatory agencies and transfer experimental results regarding the drugs to their inventors/researchers.

It is another object of the invention to disclose a remote-controlled automated system as defined above, wherein said remote robotic node additionally comprising at least one of:
a. a dosing module configured to administering said drug to the in vitro, ex vivo or in vivo systems either in the lab environment or in the natural environment of the tested organisms.
b. a monitoring module for screening of the tested animals' vital signs and physiological parameters before and during the experiment.

It is another object of the invention to disclose a remote-controlled automated system as defined above, wherein said system configured for:
a. searching and selecting of an experimental protocol for said drug-discovery experiment;
b. selecting of an experimental model according to a physical stock list and customized variables;
c. selecting of a drug or a plurality of drugs to be assessed according to a physical stock list;
d. estimating of experiment cost, time and duration according to remote node capabilities and pricing.
e. running of said experiment into the remote robotic node,
f. uploading of information
g. plotting of experimental results when requested.

It is another object of the invention to disclose a remote-controlled automated system as defined above, wherein said drug discovery experiments are non-animal experiments or animal-based experiments.

It is another object of the invention to disclose a remote-controlled automated system as defined above, wherein said non-animal experiments are selected from a group consisting of in vitro experiments, in silico experiments, ex vivo experiments, and any combination thereof.

It is another object of the invention to disclose a remote-controlled automated system as defined above, wherein organisms for said experimental models, are selected from a group consisting prokaryotes, eukaryotes, invertebrates, vertebrates, and any combination thereof.

It is another object of the invention to disclose a remote-controlled automated system as defined above wherein said prokaryotes are selected from a group consisting Escherichia coli bacterium, streptococcus bacterium, archaea and any combination thereof.

It is another object of the invention to disclose a remote-controlled automated system as defined above wherein said eukaryotes are selected from a group consisting Saccharomyces cerevisiae, Schizosaccharomyces pombe, Chlamydomonas reinhardtii, Dictyostelium discoideum, and any combination thereof.

It is another object of the invention to disclose a remote-controlled automated system as defined above wherein said invertebrates are selected from a group consisting Drosophila melanogaster or Caenorhabditis elegans.

It is another object of the invention to disclose a remote-controlled automated system as defined above wherein said vertebrates are selected from a group consisting rat, mouse, zebra fish, guinea pig, rabbit, pig, hamster and any combination thereof.

It is another object of the invention to disclose a remote-controlled automated system as defined above, wherein said experimental models are selected from a group consisting in vitro toxicity studies; genetic toxicity studies; DMPK, ADME and PK studies, in vitro efficacy studies, in vitro toxicity studies, ex vivo studies, in vivo efficacy studies, in vivo toxicity studies and any combination thereof.

It is thus one object of the present invention to disclose a method for remote-controlled automated drug testing and screening, comprising steps of:
a. obtaining a system, said system is characterized by:
   i. a selection web module for providing selected drug discovery experiments, said selection web module comprises a catalog of available experimental protocols, a list of experimental models, and a list of chemical compounds;
   ii. a remote robotic node configured to run said selected drug discovery experiments and retrieve results of these experiments;
   iii. a recording web module configured to analyze and visualize results of said selected drug discovery experiments; and
   iv. a machine learning algorithm configured to obtain said experimental results and use said results to recommend experimental parameters in a future experiment;
b. operating said system.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention

FIG. 1: A schematic representation of the invention;

FIG. 2A: A system flowchart;

FIG. 2B: An example of a web module for an experimental selection module;

FIG. 2C: Examples of an administrative preload selection from a database containing organism information;

FIG. 3: A user flowchart;

FIG. 4: Selection of a protocol, a screen snapshot;

FIG. 5: Selection of an experimental model, a screen snapshot;

FIG. 6: Selection of tested drugs, a screen snapshot;

FIG. 7: An administrative layer for standardized protocols, a screen snapshot;

FIG. 8: An example of implantation of a raw automation code for a toxicology experiment;

FIG. 9: An example of implantation of user parameters through a json code;

FIG. 10: System architecture for backend logical layer;

FIGS. 11A-11B: A scheduler monitor; FIG. 11A and FIG. 11B depict examples of either a single running experiment (FIG. 11A) or two experiments running in parallel (FIG. 11B).

FIG. 12: An IOT controller level;

FIG. 13: An example of a hardware unit;

FIG. 14: An example of a connection of a hardware unit to the controller layer through internet;

FIG. 15: An example of the visualization of the experiments: a web interface able to plot the processed data retrieved by the presenter module;

FIG. 16: A machine learning module; and

FIG. 17: Selection of experiments' sharing, a screen snapshot.

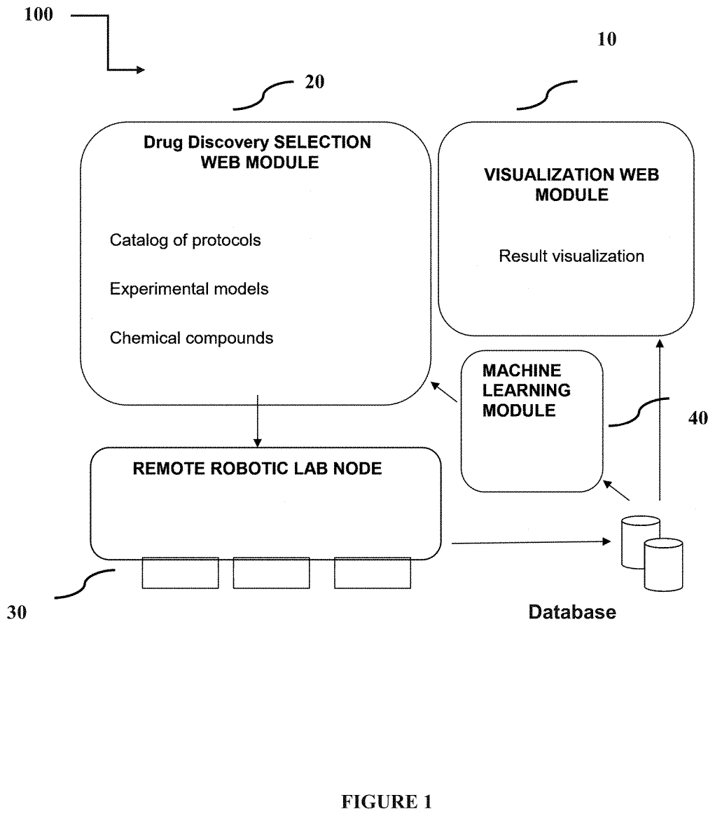

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a remote-controlled automated laboratory system for drug testing and screening.

The term JSON refers hereinafter to JavaScript Object Notation, an open-standard file format that uses human-readable text to transmit data objects consisting of attribute-value pairs and array data types (or any other serializable value). It is a very common data format used for asynchronous browser-server communication, including as a replacement for XML in some AJAX-style systems. JSON is a language-independent data format. It was derived from JavaScript, but as of 2017 many programming languages include code to generate and parse JSON-format data. The official Internet media type for JSON is application/json. JSON filenames use the extension .json.

The term PostgreSQL or Postgres, refers hereinafter to an object-relational database management system (ORDBMS) with an emphasis on extensibility and standards compliance. It can handle workloads ranging from small single-machine applications to large Internet-facing applications (or for data warehousing) with many concurrent users; PostgreSQL is ACID-compliant and transactional. PostgreSQL has updatable views and materialized views, triggers, foreign keys; supports functions and stored procedures, and other expandability.

The term MongoDB refers hereinafter to a free and open-source cross-platform document-oriented database program. Classified as a NoSQL database program, MongoDB uses JSON-like documents with schemata.

The term a compiler refers hereinafter to a computer software that transforms computer code written in one programming language (the source language) into another programming language (the target language).

The term scheduling refers hereinafter to a method by which work specified by some means is assigned to resources that complete the work. The work may be virtual computation elements such as threads, processes or data flows, which are in turn scheduled onto hardware resources such as processors, network links or expansion cards.

The term a scheduler refers hereinafter to a module which carries out the scheduling activity. Schedulers are often implemented so they keep all computer resources busy (as in load balancing), allow multiple users to share system resources effectively, or to achieve a target quality of service. Scheduling is fundamental to computation itself, and an intrinsic part of the execution model of a computer system; the concept of scheduling makes it possible to have computer multitasking with a single central processing unit (CPU). A scheduler may aim at one or more of many goals, for example: maximizing throughput (the total amount of work completed per time unit); minimizing wait time (time from work becoming enabled until the first point it begins execution on resources); minimizing latency or response time (time from work becoming enabled until it is finished in case of batch activity or until the system responds and hands the first output to the user in case of interactive activity; or maximizing fairness (equal CPU time to each process, or more generally appropriate times according to the priority and workload of each process). In practice, these goals often conflict (e.g. throughput versus latency), thus a scheduler will implement a suitable compromise. Preference is measured by any one of the concerns mentioned above, depending upon the user's needs and objectives.

In real-time environments, such as embedded systems for automatic control in industry (for example robotics), the scheduler also must ensure that processes can meet deadlines; this is crucial for keeping the system stable. Scheduled tasks can also be distributed to remote devices across a network and managed through an administrative back end.

The term Execution refers hereinafter to the process by which a computer or a virtual machine performs the instructions of a computer program. The instructions in the program trigger sequences of simple actions on the executing machine

The terms front end and back end refer hereinafter to the separation of concerns between the presentation layer (front end), and the data access layer (back end) of a part of software, or the physical infrastructure or hardware. In the client-server model, the client is usually considered the front end and the server is usually considered the back end, even when some presentation work is actually done on the server. In software architecture, there may be many layers between the hardware and end user. Each can be spoken of as having a front end and a back end. The front is an abstraction, simplifying the underlying component by providing a user-friendly interface, while the back usually handles business logic and data storage.

The terms The Internet of Things or IoT, refer hereinafter to the network of physical devices, vehicles, home appliances, and other items embedded with electronics, software, sensors, actuators, and connectivity which enables these things to connect and exchange data; thereby creating opportunities for more direct integration of the physical world into computer-based systems; and resulting in efficiency improvements, economic benefits, and reduced human exertions. IoT involves extending Internet connectivity beyond standard devices, such as desktops, laptops, smartphones and tablets, to any range of traditionally dumb or non-internet-enabled physical devices and everyday objects. Embedded with technology, these devices can communicate and interact over the Internet, and they can be remotely monitored and controlled. With the arrival of driverless vehicles, a branch of IoT, i.e. the Internet of Vehicle starts to gain more attention.

The terms Laboratory as a service, LAAS, or LaaS refer to a cloud platform with the capability to provide access to Scientific laboratory experimentation where these experiments can be provided as a service with analogy to other pure digital services on cloud like Saas (Software as a service) and Paas (platform as a service)

The terms Raspberry Pi or RasPi refer hereinafter to a series of small single-board computers developed in the United Kingdom by the Raspberry Pi Foundation to promote the teaching of basic computer science in schools and in developing countries. The RasPi is used mainly for robotics; it does not include peripherals (such as keyboards and mice) and cases. However, some accessories have been included in several official and unofficial bundles.

The term Hudson PlateCrane refers to a PlateCrane EX Microplate Handler.

The terms non-animal experiments refer hereinafter to alternatives to animal testing including tests using human cells and tissues (also known as in vitro methods), advanced computer-modeling techniques (often referred to as in silico models), and ex vivo studies, refer to experimentation or measurements done in or on tissue from an organism in an external environment with minimal alteration of natural conditions.

The term animal experiments refers hereinafter to animal testing, animal research and in vivo testing, or the use of non-human animals in experiments that seek to control the variables that affect the behavior or biological system under study.

The term in vitro (meaning: in the glass) studies refer hereinafter to animal testing which are performed with microorganisms, cells, or biological molecules outside their normal biological context.

The term ex vivo (Latin: “out of the living”) refers hereinafter to experimentation or measurements done in or on tissue from an organism in an external environment with minimal alteration of natural conditions. Ex vivo conditions allow experimentation on an organism's cells or tissues under more controlled conditions than is possible in in vivo experiments (in the intact organism), at the expense of altering the “natural” environment.

The term in vivo (Latin for “within the living”;) refers hereinafter to studies in which the effects of various biological entities are tested on whole, living organisms or cells, usually animals, including humans, and plants, as opposed to a tissue extract or dead organism.

The term experimental models refers hereinafter to experiments performed in non-human species that is extensively studied to understand particular biological phenomena, with the expectation that discoveries made in the model organism will provide insight into the workings of other organisms. Model organisms are widely used to research human disease when human experimentation would be unfeasible or unethical. Model organisms comprise inter alia prokaryotes, eukaryotes, invertebrates and vertebrates.

The term PK refers hereinafter to pharmacokinetics, a branch of pharmacology dedicated to determining the fate of substances administered to a living organism.

The term DMPK refers hereinafter to Drug metabolism and pharmacokinetics.

The term ADME refers hereinafter to an abbreviation in pharmacokinetics and pharmacology for “absorption, distribution, metabolism, and excretion”, which describes the disposition of a pharmaceutical compound within an organism. The four criteria all influence the drug levels and kinetics of drug exposure to the tissues and hence influence the performance and pharmacological activity of the compound as a drug.

The invention is related to a guided system to perform Drug Discovery experiments using a remote robotic setup. Comprising a computer assisted guided process to discover new pharmaceuticals according to their toxicity and/or efficacy, where the discovery experiments are fed into a remote automated or robotized laboratory.

The current invention is a guided system to perform drug discovery experiments using a remote robotic setup. The present invention provides systems and methods to discover new pharmaceuticals according to their toxicity and/or efficacy, where the discovery process is guided by a computer assisted system and performed in a remote laboratory.

The present invention provides systems and methods to discover new pharmaceuticals according to their toxicity and/or efficacy, where the discovery process is guided by a computer assisted system and performed into a remote laboratory. The system has a plurality of processing modules consisting in (1) A web module for selection of Drug Discovery experiments containing a catalog of available experimental protocols, a list of experimental models, and a list of chemical compounds (2) A remote robotic node able to run the selected experiments and retrieve data (3) A web module for visualization of experimental results (4) A machine learning algorithm able to get metadata from experimental results and use this data to recommend experimental parameters in a future experiment.

The current invention discloses a system to assess and select drugs by their toxicity, pharmacokinetics and efficacy using in vitro, ex vivo or in vivo systems.

The current invention is further described by: a remote-controlled automated laboratory system for drug testing and screening (100), the system characterized by:
a. a selection web module (20) for providing selected drug discovery experiments, said web module comprises a catalog of available experimental protocols, a list of experimental models, and a list of chemical compounds;
b. a remote robotic node (30) configured to run the selected drug discovery experiments; and retrieve results of these experiments;
c. a recording web module (10) configured to analyze and visualize results of the selected drug discovery experiments; and
d. a machine learning algorithm (40) configured to obtain the results of the experimental results and use this data to recommend experimental parameters in a future experiment.

The uniqueness of the current invention is the ability of the system to select the preferred experimental designs and protocols for a new drug or pharmaceutical agent according to available data, data base captured during previous experiments, and according to guidelines provided by regulatory agencies such as FDA NIH, EMEA and similar agencies, and to perform the selected experiments robotically and remotely. The current invention is capable to perform the selected experiments, either using in vitro systems (such as cell cultures, microorganisms), ex vivo systems (on excised organs such as skin) and even using in vivo systems of either small organisms like C. elegance Zebra fish or Drosophila or phylogenetically-higher organisms such as rats and dogs, which are implanted with electrodes or sensors or non-invasive devices.

Experimental Models, Organisms The current system is used for selection of drugs using organisms experimental models; non-limiting examples of model organisms are listed in the following paragraphs:
a. Prokaryotes: The most widely studied prokaryotic model organism is Escherichia coli (E. coli), which has been intensively investigated for over 60 years. It is a common, gram-negative gut bacterium which can be grown and cultured easily and inexpensively in a laboratory setting. It is the most widely used organism in molecular genetics, and is an important species in the fields of biotechnology and microbiology, where it has served as the host organism for the majority of work with recombinant DNA. Examples of Prokaryotes include inter alia also Streptococcus and Archaea
b. Eukaryotes: Simple model eukaryotes include baker's yeast (Saccharomyces cerevisiae) and fission yeast (Schizosaccharomyces pombe), both of which share many characters with higher cells, including those of humans. For instance, many cell division genes that are critical for the development of cancer have been discovered in yeast. Chlamydomonas reinhardtii, a unicellular green alga with well-studied genetics, is used to study photosynthesis and motility. C. reinhardtii has many known and mapped mutants and expressed sequence tags, and there are advanced methods for genetic transformation and selection of genes Dictyostelium discoideum is used in molecular biology and genetics, and is studied as an example of cell communication, differentiation, and programmed cell death.
c. Invertebrates: Among invertebrates, the fruit fly Drosophila melanogaster is famous as the subject of genetics experiments. The fruit flies are easily raised in the lab, with rapid generations, high fecundity, few chromosomes, and easily induced observable mutations. The nematode Caenorhabditis elegans is used for understanding the genetic control of development and physiology. It was first proposed as a model for neuronal development by Sydney Brenner in 1963, and has been extensively used in many different contexts since then. C. elegans was the first multicellular organism whose genome was completely sequenced, and as of 2012, the only organism to have its connectome (neuronal “wiring diagram”) completed.
d. Vertebrates: Among vertebrates, guinea pigs (Cavia porcellus) were used by Robert Koch and other early bacteriologists as a host for bacterial infections, becoming a byword for “laboratory animal,” but are less commonly used today. The classic model vertebrate is currently the mouse (Mus musculus). Many inbred strains exist, as well as lines selected for particular traits, often of medical interest, e.g. body size, obesity, muscularity, and voluntary wheel-running behavior. The rat (Rattus norvegicus) is particularly useful as a toxicology model, and as a neurological model and source of primary cell cultures, owing to the larger size of organs and suborganellar structures relative to the mouse, while eggs and embryos from Xenopus tropicalis and Xenopus laevis (African clawed frog) are used in developmental biology, cell biology, toxicology, and neuroscience. Likewise, the zebrafish (Danio rerio) has a nearly transparent body during early development, which provides unique visual access to the animal's internal anatomy during this time period. Zebrafish are used to study development, toxicology and toxicopathology, specific gene function and roles of signaling pathways.

### Experimental Models:

In vitro toxicity studies comprise inter alia:

a. Genotoxicity testing

b. Skin irritancy/corrosivity testing

c. Eye irritancy/corrosivity testing

d. Skin sensitization

e. Cytotoxicity testing

f. In vitro carcinogenicity

g. Endocrine disrupter screening

h. Vaccine safety/efficacy evaluation

i. Antimicrobial development/efficacy

j. Microbial Pest Control Agent safety assessment/quality control

k. Bacteriology services for clinical trials

Genetic toxicity studies comprise inter alia:

a. Ames test

b. Mouse lymphoma assay

c. Chromosome aberration test

d. In vitro micronucleus test

e. In vivo micronucleus test

f. Unscheduled DNA synthesis test (in vitro and in vivo)

g. Comet assay (in vitro and in vivo)

h. mouse lymphoma assay,

i. in vitro mammalian chromosome aberration test in human lymphocytes

j. in vitro skin and eye irritation/sensitization assays (BCOP, Episkin)

DMPK, ADME and PK studies comprise inter alia:

a. In-vitro metabolism and Drug-Drug Interaction assessment specialist group

b. Rapid screening PK

c. Bioavailability and Bioequivalence

d. Toxicokinetics

e. Blood-brain barrier transfer

f. Absorption studies

g. Tissue distribution

h. PK/PD modelling

i. Formulation comparison

j. Food effect

k. Non-compartmental and compartmental pharmacokinetics

l. Surgical models

m. Multiple routes for test item administration

n. Cassette dosing

o. Tissue, CSF and urine sampling

p. Bioanalysis

q. Cardiotoxicity liability testing in cardiac ion channels, including Herg assay

r. Biomarker assay development and qualification—safety and efficacy markers

s. Kinetics in human and animal hepatocytes

t. Membrane permeability (CaCO-2 cells)

u. Metabolic stability in human liver microsomes or hepatocytes

v. CYP inhibition

w. Protein binding in human plasma

In vivo toxicity studies (safety pharmacology)

a. Cardiovascular

b. Respiratory assessment

c. Central nervous system

d. Single and repeat dose in vivo non-clinical pharmacokinetics

e. Electrophysiology

f. Follow-on studies

In vivo efficacy studies comprise inter alia:

a. respiratory inflammation models:
The house dust mite model of chronic allergic inflammation (mouse)
Antigen-induced pulmonary inflammation (ovalbumin sensitized brown Norway rat mouse or guinea pig)
LPS induced non-allergic pulmonary inflammation (mouse, rat, guinea pig; also primate in our US facility)
Cigarette smoke-induced pulmonary inflammation (mouse)
Bleomycin-induced lung fibrosis (rat)
Bronchoconstriction/bronchodilator studies in conscious and anesthetized animals (rodent, guinea pig and dog)

b. routes of delivery
Inhalation delivery optional
Aerosol and dry powder
Unique in-house developed system for dry powder delivery using small amounts of test material

c. Gastrointestinal models:
Emesis/anti-emesis (ferret)
GI motility (rodent; large animal planned for development)
IBD models (rodent; primate planned for development)
Feeding/dietary models (rodent)

d. Anti-infective models:
Wound healing (+/− MRSA infection) (rodent, rabbit, pig)
Influenza—tissue burden and biomarker endpoints
Host resistance

e. Cardiovascular models:
Heamodynamics and electrocardiology (rodent and large animal)
Echocardiography in development for rodent and large animals
Ion channel electrophysiology
Bioanalysis and biomarker (translational) assessment

f. Oncology models:
Tumor Implants
Human xenografts in nude athymic mice
Orthotopic and ectotopic implantation
Leukemia models (NOD/SCID)
Induced Metastasis Models
Orthotopic and footpad implantation
Xenograft in Humanized Mice
Syngeneic Tumor Models
Immune and Inflammatory Disease Models
Arthritis
CIA (collagen induced arthritis)
SCW Streptococcal Cell Wall Arthritis
Mouse Adjuvant Arthritis
Cytokine Analysis, Histopath, X-ray analysis, Joint Swelling
Chronic Joint Inflammation
Mouse Type II Collagen (CIA) and SCW Arthritis
Mouse Adjuvant Arthritis
Cytokine Analysis, Histopath, X-ray analysis, Joint Swelling
Acute Paw Inflammation
Mouse Carrageenan and Oxazolone Paw Edema
Mouse Zymosan Paw Edema
Cytokine Analysis
Acute Air Pouch Inflammation
Mouse & Rat (Carrageenan, Chemoattractant, TNF-a, IL-1b, Superantigens)
Cytokine Analysis, Cell Number, and Cell Phenotyping (Cytospin & FACS)
Acute Inflammation
Mouse LPS-induced inflammation (local and systemic)
Mouse LPS/D-Gal-induced mortality
Cytokine Analysis
Acute Rhinitis module Dermal Irritation (Draize Modified Scoring) Systemic InflammationDelayed Type Hypersensitivity (DTH)
Mouse air pouch model with chemoattractants, superantigens, and toxins
Mouse intraperitoneal migration model with Chemokines, superantigens, toxins and Oxazolone
Mouse and Minipig Th1 and Th2 DTH and ACD skin migration model
Cytokine Analysis, Cell Migration, Ex Vivo Cell Proliferation
Monosodium-Urate (MSU) crystals induced mouse neutrophil migration
Mouse air pouch model with chemoattractants, superantigens, and toxins
Mouse intraperitoneal migration model with Chemokines, superantigens, toxins and Oxazolone
Mouse and Minipig Th1 and Th2 DTH and ACD skin migration model
Cytokine Analysis, Cell Migration, Ex
Chronic CNS Inflammation
Mouse Experimental-Induced Encephalomyelitis (EAE) (Acute B6 Mice, SJL, Biozzi Mice)
Spontaneous EAE mouse B6/RAG/TR model
Cytokine Analysis, Cell Proliferation
Gastrointestinal Inflammation/Irritation (colitis models)
Colonic Inflammation: Mouse Dextran Sulfate and Oxazolone Models
Acute Gastric Irritation in mice (4 hr)
3-day Intestinal Irritation in mice (72 hr)
Cytokine Analysis, colon assessment and histopath
Asthma and COPD
Mouse Ovalbumin asthma model
Mouse Cockroach asthma model
Mouse PPE induced COPD
Smoke induced COPD
Cytokine Analysis, cell differential count in broncho-alveolar lavages
Liver and lung fibrosis models
Liver fibrosis (CCL4 and DNFB induced) and Lung fibrosis (Belomycin induced)
Mouse Ovalbumin asthma model/Dermatitis models
Atopic dermatitis in NcNGA transgenic mice
DNFB induced dermatitis in mice and pigs
Engraftment of human Psoriatic skin in mice
IL-12/lps-induced human like psoriasis in scid mice

g. Diabetes Type 1 and 2
Mouse
Rat
MiniPig

h. Wound Healing
Mice
Rat
MiniPig (deep wounds, long term)

i. Central Nervous System
BTS Research core competency in the area of CNS is based on IND enabling study requirements.
Behavioral Screening Open Field
Morris Water Maze
Paw Strength (Front or back limps)
Roto-Rod
Epilepsy
Neurodegeneration
Parkinson's
Huntington's
Alzheimer's
Epilepsy
Spinal Cord Injury
Glioblastoma
Custom CNS Models
Behavioral Screening
Open Field Activity
Morris Water Maze
Roto-Rod
Beam Walk
Rotometer
Paw Placing

j. Various Models
Acute and chronic CC14 and DMN liver fibrosis (mice, rats)
Acute or chronic-repeated short and long term infusion/dosing
ADME
ADME-NHP, Rodent, Dog, Pig
Air pouch
Air pouch model on rats (inflammation)
Antibody production
Asthma Ovalbumin Induced
Bile duct cannulated colony
Bladder manipulation
Brain receptor occupancy: Mouse, Rat
Brain receptor occupancy: Rat
Calvarial defect: Rat
Canulated rat infusion PK
Cardiovascular
Cecal Ligation
Cecal ligation: Rat
CIA on murine
CLP-induced sepsis
CNS (some models)
Colitis
Colitis DSS induced
Collagen Induced Arthritis CIA
Contact hypersensitivity (˜delayed type hypersensitivity)
COPD Elastase-Collagenase and smoke induced.
db/db mouse dermal wound
Dermatitis
Diabetic
Diabetic models on rats and pigs
Diet-induced obesity (<6 months)
Diet-induced type II diabetes
DIO feeding study: Mouse
Dorsal and ventral spinal nerve electrophysiology
DTH Oxazalone and DNFB induced
DTH on murine and pigs
EAE (experimental autoimmune encephalomyelitis)
EAE MOG+PT induced
EAE PLP induced
EAE on murine
Full thickness dermal wound healing: Rabbit
Functional Observational Battery
Gastric emptying
Genetic models of obesity (mouse and rat)
Genetic models of type II diabetes (mouse and rat)
Hepatic fibrosis CC14 induced
Hepatic fibrosis DMN induced
Hollow fiber cell assay: Mouse
Infection Model (E. coli and S. aureus resistant strains)
Insulin tolerance test (IP)
Insulin tolerance test (IV)
Intra Occular Pressure measurements
Kidney Disease
IV dose MOLT4 for leukemia model
IV injection of labeled-neutrophils in rabbits and assessing chemokine-mediated migration
Laser Doppler blood flow
Liver Disease
Liver perfusion (Cardiac flush and Infusion pump flush)
LPS and Galactosamine inflammation model
LPS lethality
Lung inflammation model, cockroach antigen
Lung Metastasis (i.e. B16-F10)
Lupus model
Mass balance
Models of hyperlipidemia
Mouse OVA asthma
Mouse xenograft models
Neurotox Testing Batteries
Obesity
Ocular
Occular and opthalmic models
Oral glucose tolerance test
Organ cell inoculation (liver and kidney)
Orthotopic and ectopic bone healing: Rabbit
Orthotopic and ectopic bone healing: Rat
OxyMax calculation of fatty acid and carbohydrate oxidation rates
OxyMax open calorimetry system (acute and sub-chronic)
Parkinson's
Passive cutaneous anaphylaxis
Patch application
Peripheral nerve electrophysiology
PK
Pulmonary and hepatic fibrosis: Mouse
Pulmonary/hepatic fibrosis: Rat
Radiation Exposure
Rat adrenalectomy
Rat ICV studies
Rectus muscle pouch: Rat
Repeated long term IV infusion
Rodent dermal inflammation
Rodent models of diabetes (type I and type II)
Rodent vascular permeability
Rotarod
Smoke inhalation in mice
Spinal ligation: Rat
Spinal surgeries
Stem Cell
Sterotaxis
Stifle joint injection and aspiration of sinovial fluid: Dog
Stroke
Subcutaneous pouch: Rat
TNF+/− galactosamine lethality
Tox—small and large animal with daily dosing
Ulna defect model (long bone): Rabbit
Vein graft (femoral artery/vein): Rat
Von Frey hair sensory testing
Water maze
Wound healing Model on rats and Pigs
Wound healing with laser Doppler testing
Xenograph Tumor Model

The current invention additionally discloses using a machine learning module. Additionally, the system can use blockchain technology in order to enable secured information available from regulatory agencies and transfer experimental results regarding the candidate drugs to their inventors/researchers.

The system may have several other modules such as:
a. a dosing module—for administrating the drugs to the in vitro, ex vivo or in vivo systems either in the lab environment or in the natural environment of the tested organisms.
b. a monitoring module for screening of the tested animals' vital signs and physiological parameters before and during the experiment.

The system also enables to perform double blind experiments, with minimal intervention of the experimenter.

The system of the current invention is capable to test drug toxicity using systems selected from in vitro, ex vivo and in vivo system in cells, microorganisms, invertebrates and vertebrates. The system of the current invention is also capable to test drug efficacy_using systems selected from in vitro, ex vivo and in vivo system in cells, microorganisms and invertebrates but not in vertebrates.

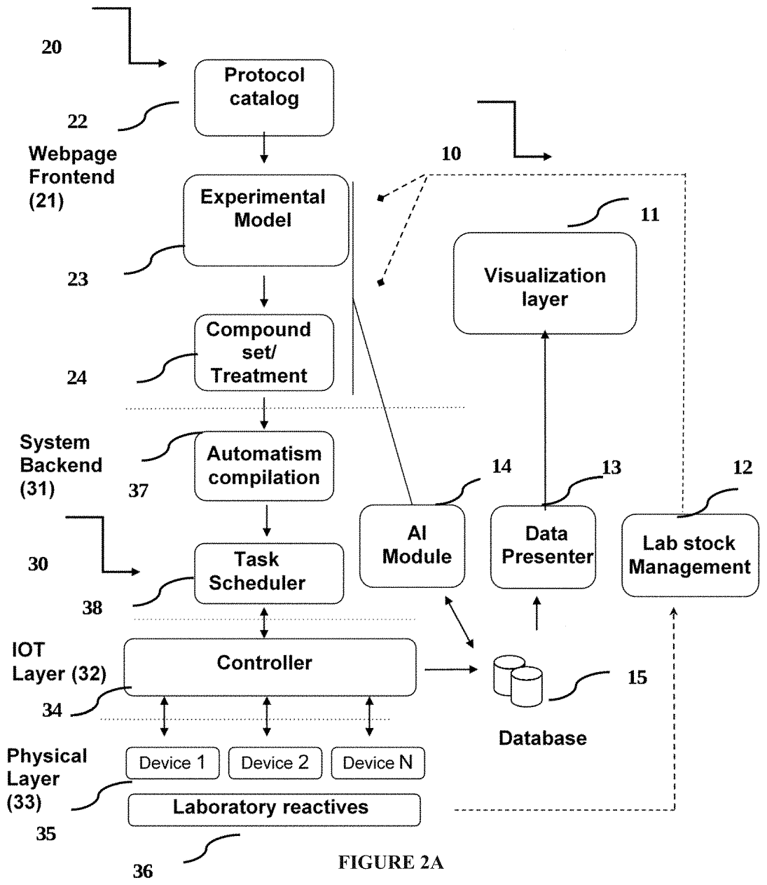

Each modules of the system comprises several layers, which operate as follows (FIG. 2A):

a. a selection web module (20) comprising:
(i) A webpage frontend layer (21); comprising several modules: a protocol catalog (22), an experimental model selection module (23), and compound set/treatment module (24).
   Experimental model selection module (23), comprises a web module (see FIG. 2B) able to select the desired organism/experimental model by the user.
   This web-module (frontend) obtains the information from a Database Table containing organism name, current stock, expiration date, and associated Filler Device (device in charge to dispense the organisms/experimental model). This information is preloaded, for example, by administrators of a cloudlab platform (see FIG. 2C) and stock is consumed as used.
(ii) Administrative layer (not shown)—Standardized experimental protocols are managed in an administrative layer where title, plot, protocol abstract, and parameters able to configure by the user are detailed (located between selection web module and remote robotic)

b. a remote robotic node (30) comprising:
(i) A system backend (31), comprising automatization compilation module (37); task scheduler module (38).
   Compilation module (37) is in charge of processing the .txt “Macro code” language, filling the variables according to user selection criteria. It fills:
      the timing parameters selected graphically by the user at frontend;
         the corresponding device to dispense the selected experimental model (DISPENSER); and
      the corresponding compounds location ($STATION) to collect samples.
   Compilation module (37) runs in a backend once the user has accepted to start the experiment, and it passes the compiled instructions to the task scheduler (38).
   Task scheduler (38) is in charge of fitting the automation needs with the availability of hardware resources.
   It works by creating a timing allocation table for the specific automatism, and comparing the availability of resources from present to future in 1 minute timeblocks until fitting.
   It determines the precise timing of start of the experiment. It selects between duplicated devices of which one will be used for the experiment according to availability, and it reserves the devices for the corresponding Experimental ID.
   The output of the Task scheduler is the complete automation code including Device ID and absolute time of execution. This complete code will be read by the Executor module (further described herein).
(ii) An IOT layer, comprising a controller (34); interconnected to data base (15)
(iii) Physical layer comprising at least one device (35) and laboratory reactives (36); interconnected with lab stock management (12).

c. a visualization web module (10), configured to analyze and visualize results of the selected drug discovery experiments; comprising AI module (14);) data presenter (13); lab stock management (12) and a visualization layer (11).

The function of each part of the system are described in the following paragraphs:

Webpage frontend layer (21): The webpage frontend is a user interface implementation. It is a web layer containing a searchable list of cards, representing experimental protocols to be executed. Where each card has associated a high level code of hidden instructions with fixed and variable parameters.

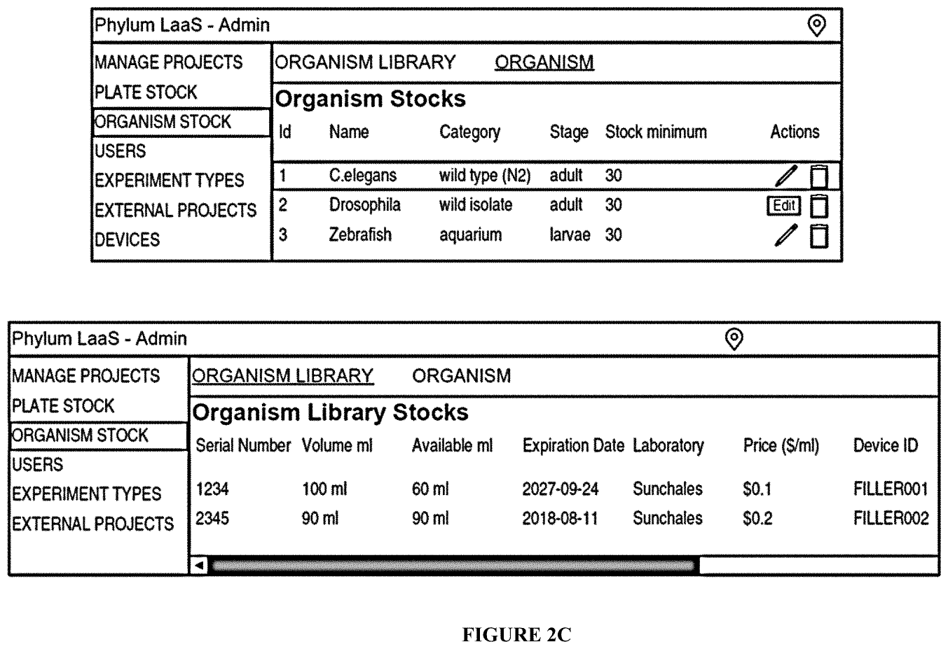

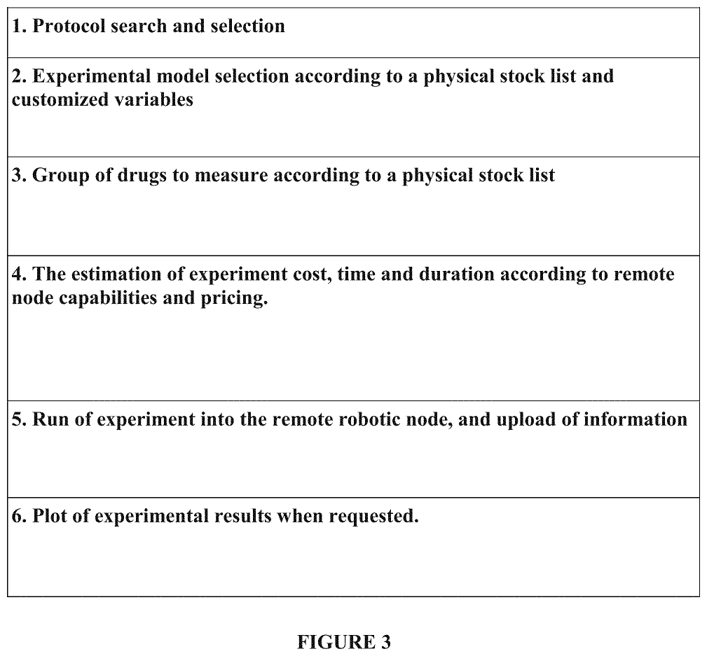

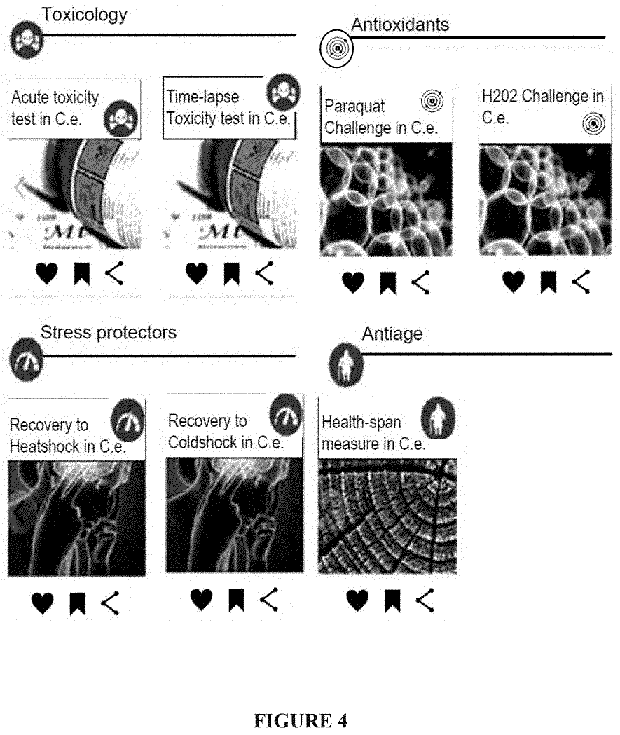

The flowchart comprises a configuration step of:
a. the selection of a protocol in a list of them presented in graphic cards (FIG. 4);
b. the selection of an experimental model, and customization of limited set of protocol variables (FIG. 5); and
c. the selection of drugs to test (FIG. 6).

As shown here, the complexity of programming code is hidden to the user, and no line programming need to be done. In order to make this feature possible, all the machine code information is managed internally in an administrative layer controlled by the hosting laboratory offering the solution.

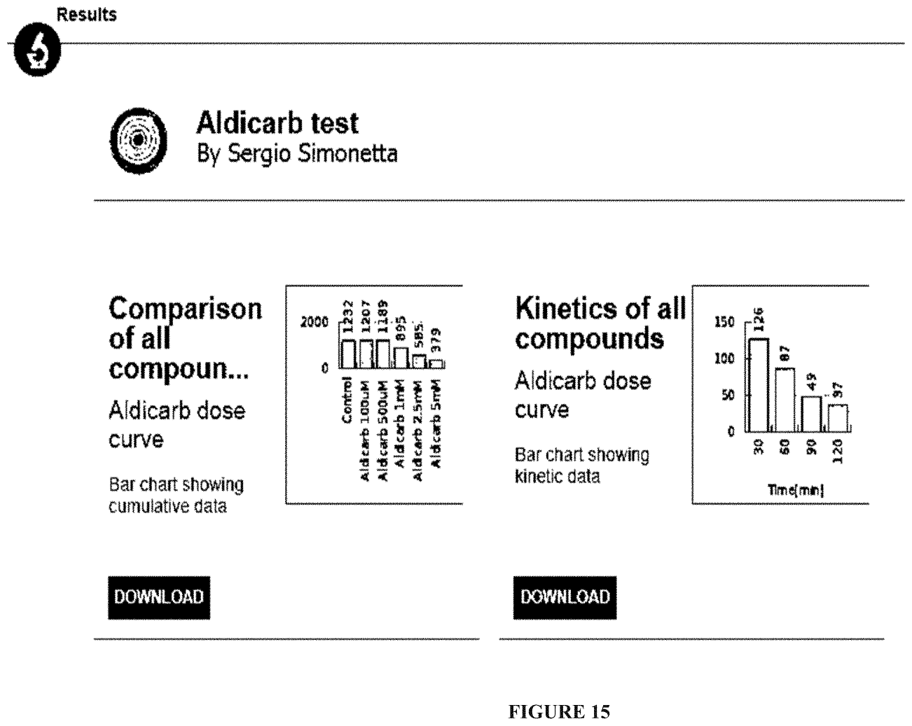

Visualization of experiments: visualization of experiments is performed using the following modules:
a. A Presenter module (13), consisting in a processing algorithm able to retrieve the data from the database of experiments (15), and process it according to a high level configuration of the associated protocol.
b. Web interface able to plot the processed data retrieved by the Presenter module, (FIG. 15).

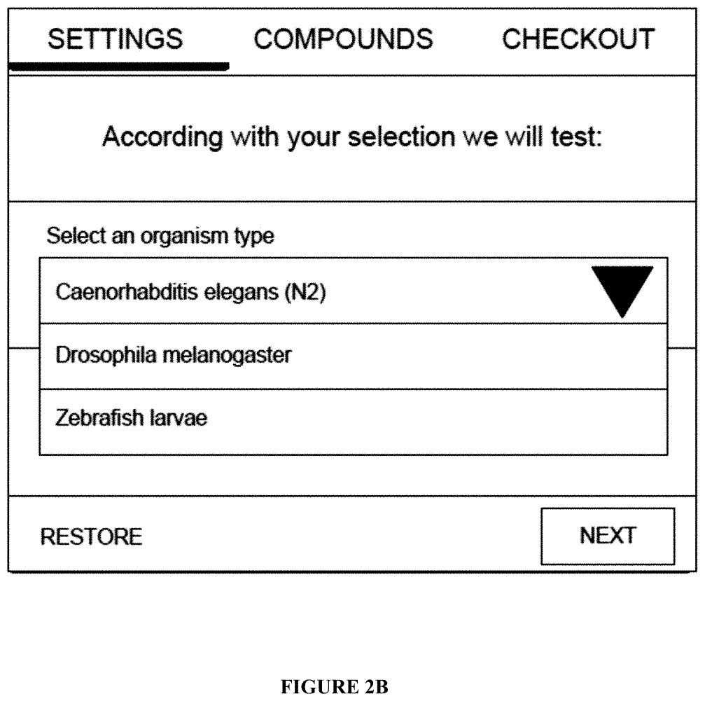

FIG. 3 depicts the user's flow chart, comprising the flowing steps:
a. Protocol search and selection
b. Experimental model selection according to a physical stock list and customized variables
c. Group of drugs to measure according to a physical stock list
d. The estimation of experiment cost, time and duration according to remote node capabilities and pricing.
e. Run of experiment into the remote robotic node, and upload of information
f. Plot of experimental results when requested.

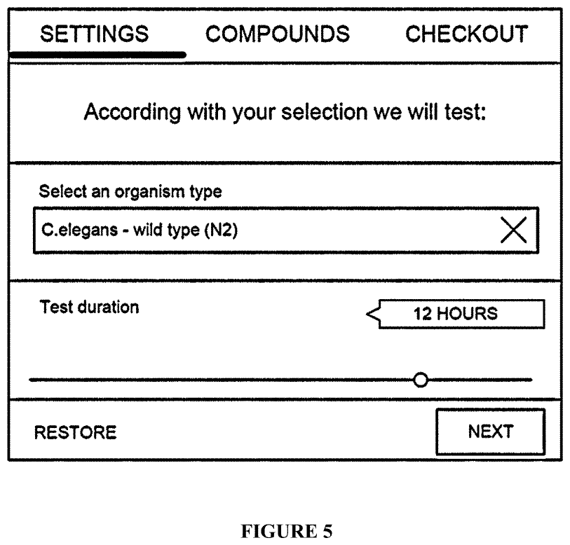

Administrative layer: The administrative layer is used for standardized protocols. Standardized experimental protocols are managed in an administrative layer where title, plot, protocol abstract, and parameters able to configure by the user are detailed. (FIG. 7)

Raw Automation code: The Raw automation code is the automatism associated to a standardized protocol and uploaded in the administrative layer.

In a general form this is a list of macro instructions (legible by humans) containing a sequence of time defined actions, associated device type, and variables to be completed by the system.

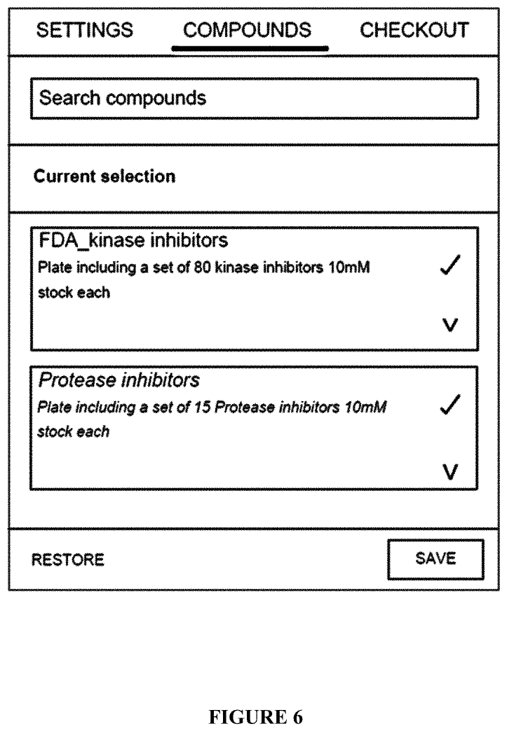

An example of implementation of this code for a toxicology experiment is depicted in FIG. 8.

In this case, a plate containing the desired compounds to test is taken by a robotic “ARM” from $STATION (station ID to be completed by the system at compilation time according to user selection), this plate is put in a microorganism dispenser, and later on is carried to a waiting STATION for a time interval ($INTERVAL to be completed by the system at compilation time according to user selection). Once the incubator time finish, the plate is taken from $STATION by the robotic ARM and the readout is read for 30 minutes into READER(A) (device to be allocated by the compiler according to available resources). After the read the plate is again taken and drop to a Trash position.

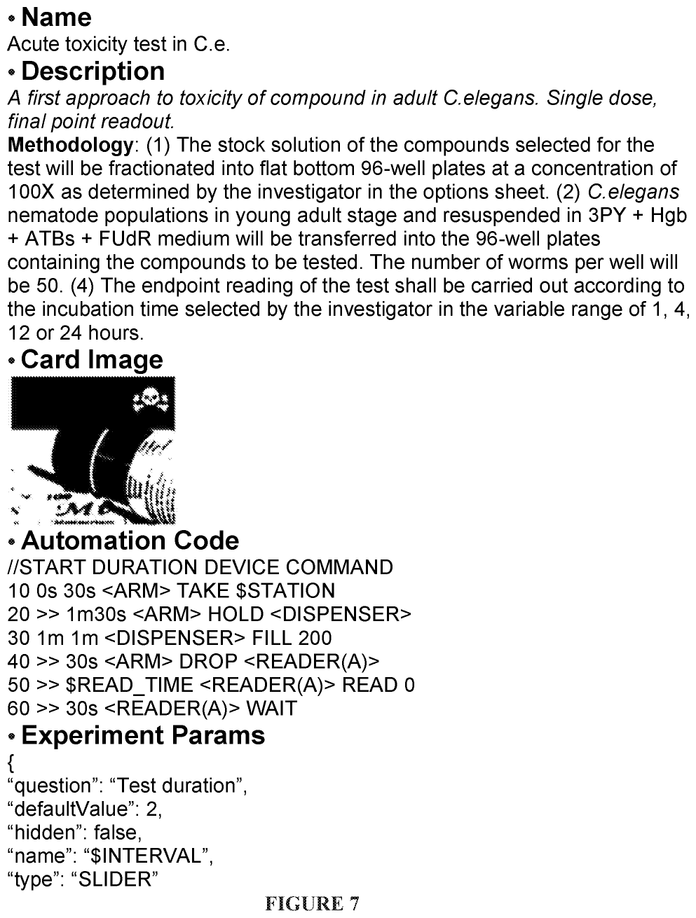

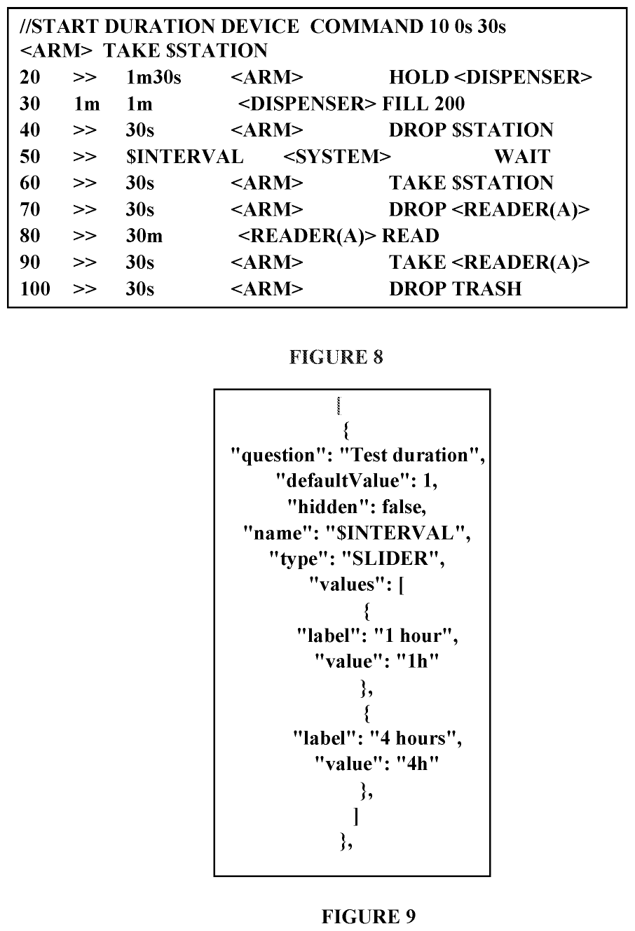

User parameters: An implementation of user parameters has been implemented through a json code such as shown in FIG. 9. The frontend webpage is in charge to use this data for configuration. System architecture for this implementation is depicted in FIG. 10.

### System Backend Logical Layer (31).

Compiler module (37): This module fill the automation code variables using the selection performed by the user, and prepare the high level experimental protocol for interpretation by the remote hardware units.

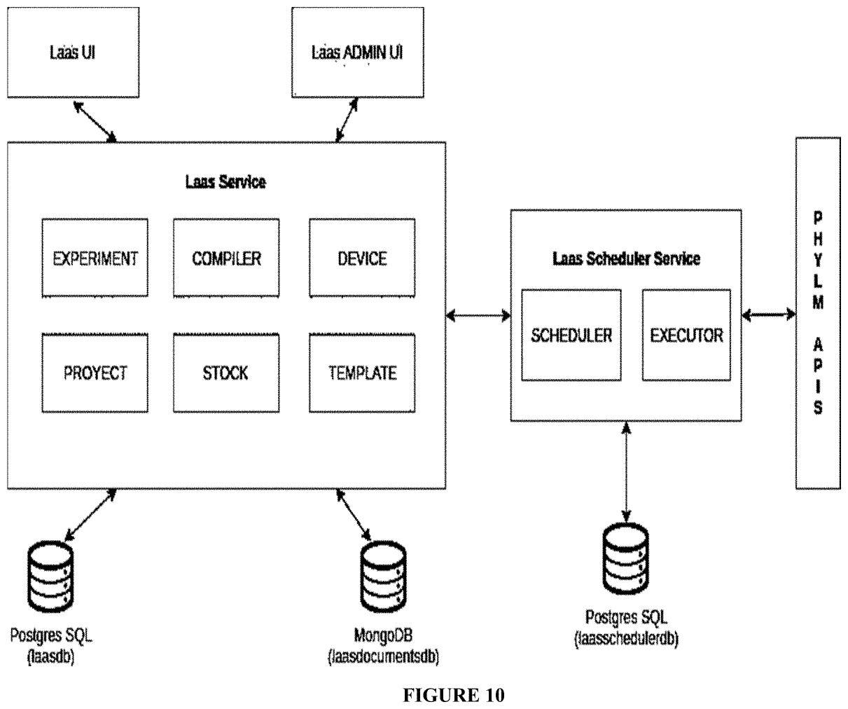

Scheduler module (38): The scheduler module fits the list of tasks into a schedule according to availability of the hardware units associated to the experimental protocol in a sequential time-frame space (FIG. 11). The drug discovery experiments can be run in parallel for saving time and cost. FIG. 11 depicts two examples:

FIG. 11A depicts pipeline of one single experiment (ID3162) running. Filled rectangles shows the occupation of each physical device in time.

FIG. 11B depicts example of two experiments running in parallel (Experiment ID3162 and ID3163). The assignment of free devices by the scheduler to have the capability to run multiple experiments in parallel is shown.

Executor module (not shown): The executor module communicate in a time synchronized manner the instructions to the hardware controller interface.

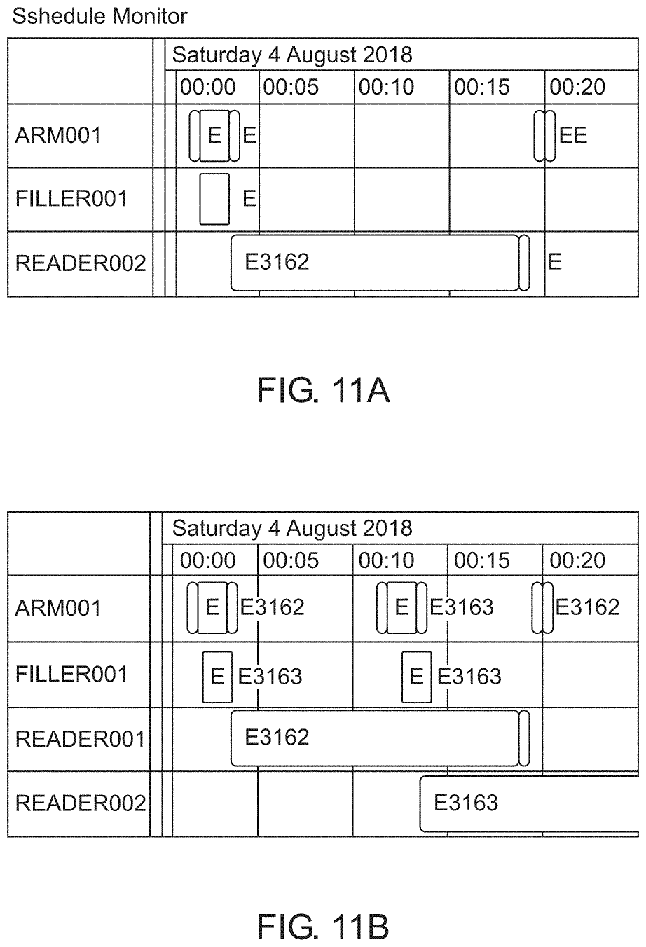

IOT controller layer (34): The controller layer is the interface in charge to receive the instructions from the backend logical layer and interact with hardware devices (FIG. 12). This module manage the instructions to specifically pass to each device the corresponding instruction to run.

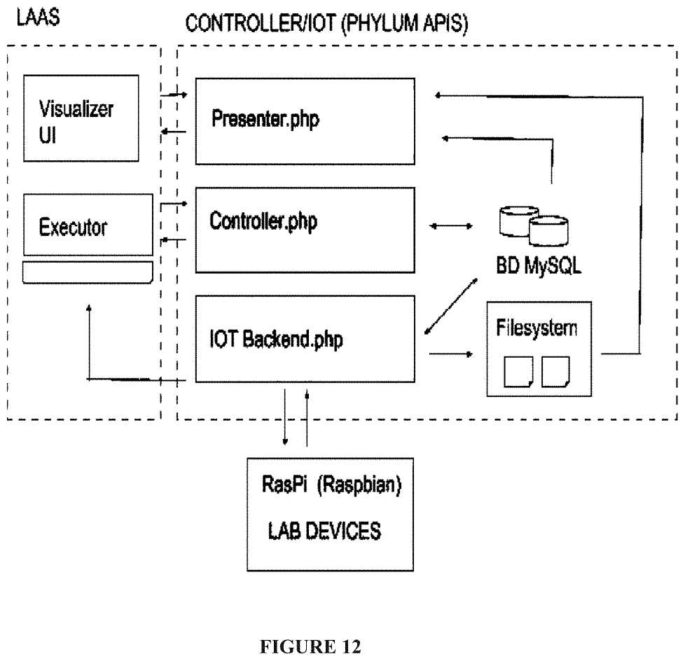

Devices (35): One or more hardware units including: at least one robotic arm able to transfer assay receptacles (microplates) from one location to another, and connected to the internet, where the movement is commanded by the controller layer. In the present application, the system has being designed using a robotic ARM HUDSON (USA) Platecrane, coupled by a RS232 port to a Raspberry Py. Each RasPi poses a program designed in C++ to internet poll to the controller server every 10 seconds to ask for new instructions to run. (FIG. 13).

One or more hardware units connected to the controller layer through internet able to acquire quantitative experimental data (absorbance, luminescence, imaging, electrophysiology, or any other measure) from the assay receptacles (microtiter plates). Where the initiation of measurement is controlled by the Controller layer, and data is transferred to controller layer.

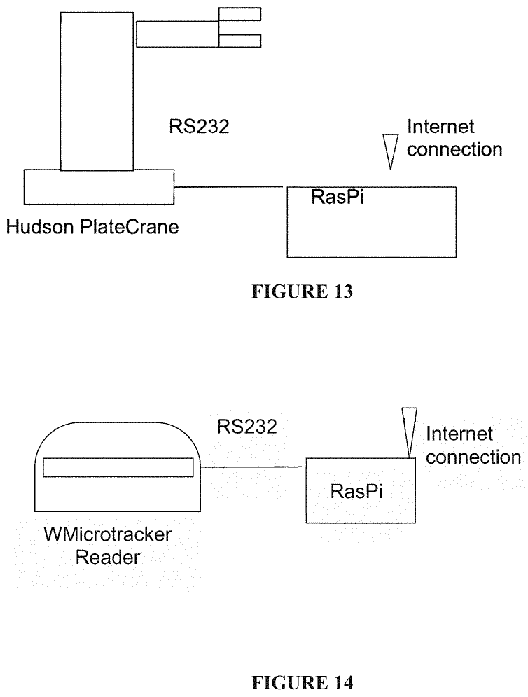

In the present application, a WMicrotracker unit (Phylumtech SA) has been coupled by a RS232 port to a Raspberry Py. Each RasPi poses a program designed in C++ to internet poll to the controller server every 10 seconds to ask for new instructions to run. (FIG. 14).

Experimental result database and filesystem (15) is located at the cloud server able to save the data acquired by the acquisition systems located in the robotic platform.

Machine learning Module Machine learning module is in charge to give recommendations to the user concerning experimental parameters to configure.

In practical terms these recommendations are presented as “natural language messages” and “statistical information based on previous experience/information of the system”.

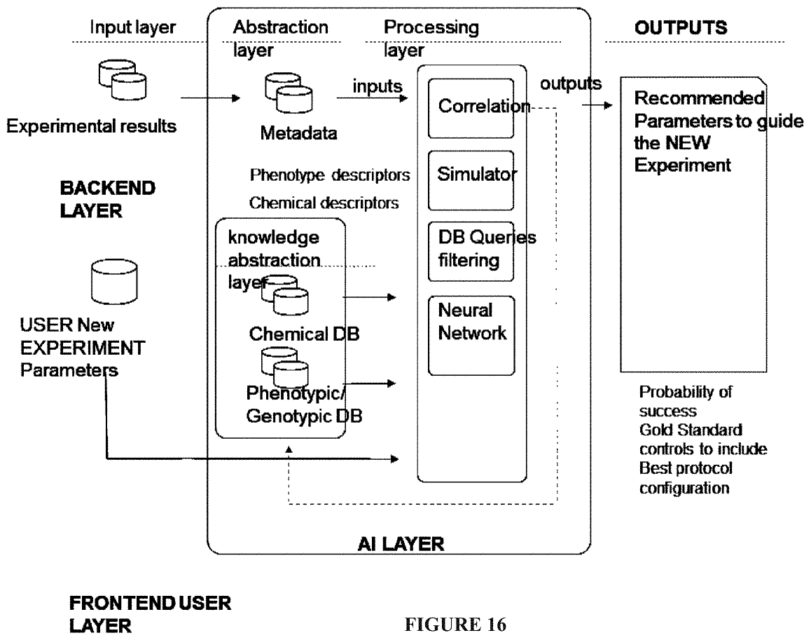

AI module (14) is comprised of a MacroData database fed by descriptors of each cloudlab experimental result, a Knowledge Database internally generated, plus one or more data integration submodules implemented as data correlation algorithms, DB queries, montecarlo simulators and neural networks. (FIG. 16). The AI module (14) is fed by public or shared result information within the cloud lab system, plus private data from the current user. The customers are rewarded in some way for sharing that data.

User management: The system allows the capability to be used by multiple users with login. Data is maintained private or public according to user selection.

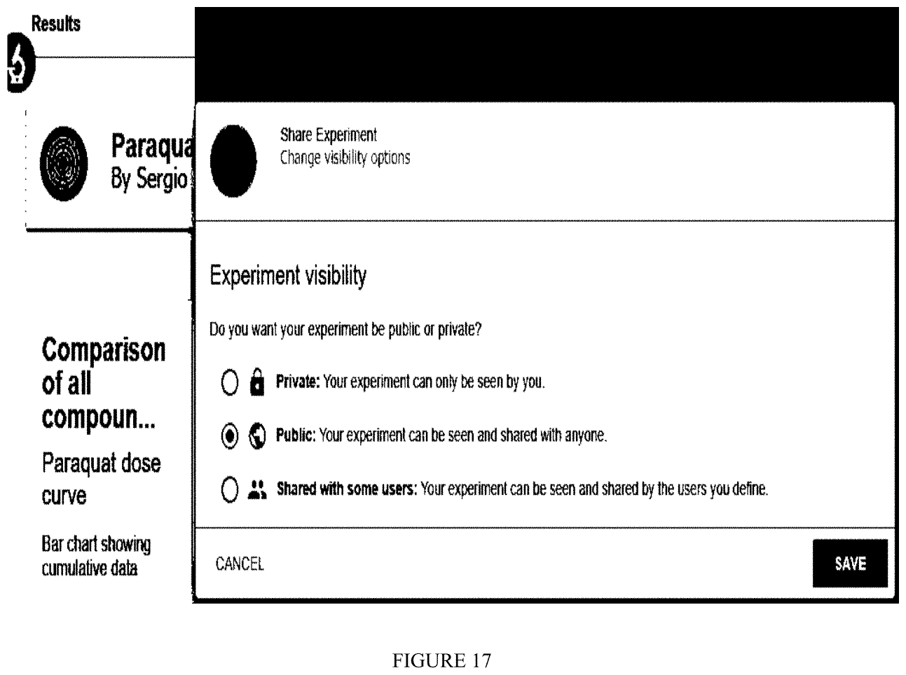

Experiments sharing: The user has the capability to set the public property of his experiment. The experiment can be private, shared with some users or public (FIG. 17).

Sharing rewards: This is a system/method for sharing confidential results in cloud based laboratories. Cloud based laboratories (such as LAAS or SCiAAS) lets run multiple experiments by different users in parallel. Confidentiality of information and data encryption are standards of this kind of systems. With the accumulation of data coming from experiments, many results/information could be of interest to users without the possibility to know each other than the experimental data for one assay is already available in hands of another user. The possibility of sharing this information is presented in order to avoid the need to run a wet or in silico experiment, to save time, physical resources and money. However, as the information is confidential a method reliable for both part must be designed. In this patent we present a method for sharing information based on rewards. An example of implementation in a wet drug discovery laboratory is shown.

1. A remote-controlled automated system for drug testing and screening, said system characterized by:
a. a selection web module for providing selected drug discovery experiments, said selection web module comprises a catalog of available experimental protocols, a list of experimental models, and a list of chemical compounds;
b. a remote robotic node configured to run said selected drug discovery experiments and retrieve results of these experiments;
c. a visualization web module configured to analyze and visualize results of said selected drug discovery experiments; and
d. a machine learning algorithm configured to obtain said experimental results and use said results to recommend experimental parameters in a future experiment.

2. The system of claim 1, wherein said system configured to run a plurality of said drug discovery experiments at the same time period.

3. The system of claim 1, wherein said system configured to share said experimental results confidentially among users of said system.

4. The system of claim 3, wherein said users are rewarded for sharing said experimental results among users of said system.

5. The system of claim 1, wherein said selection web module comprises:
a. a webpage frontend layer; said frontend layer comprises several modules: a protocol catalog, an experimental model selection module), and compound set/treatment module; and
b. an administrative layer interconnected with said frontend layer, configured to manage standardized experimental protocols according to parameters set by user of said system.

6. The administrative layer of claim 2, wherein said parameters comprise title, plot, protocol abstract, and any combination thereof.

7. The system of claim 1, wherein said remote robotic node comprises
a. a system backend, said system backend comprising an automatization compilation module); a task scheduler module;
b. an IOT layer, comprising a controller; said controller interconnected to a data base; and
c. a physical layer comprising at least one device, and laboratory reactives (36); said physical layer is interconnected with lab stock management.

8. The system of claim 1, wherein said visualization web module comprises:
a. an AI module;
b. a data presenter;
c. a lab stock management; and
d. a visualization layer.

9. The system of claim 1, wherein said system additionally comprises a blockchain module configured to transaction of secured information available from regulatory agencies and transfer experimental results regarding the drugs to their inventors/researchers.

10. The system of claim 1, wherein said remote robotic node additionally comprising at least one of:
a. a dosing module configured for administering said drug to the in vitro, ex vivo or in vivo systems either in the lab environment or in the natural environment of the tested organisms; and
b. a monitoring module for screening of the tested animals' vital signs and physiological parameters before and during the experiment.

11. The system according to claim 1, wherein said system configured for:
a. searching and selecting of an experimental protocol for said drug-discovery experiment;
b. selecting of an experimental model according to a physical stock list and customized variables;
c. selecting of a drug or a plurality of drugs to be assessed according to a physical stock list;
d. estimating of experiment cost, time and duration according to remote node capabilities and pricing;
e. running of said experiment into the remote robotic node;
f. uploading of information; and
g. plotting of experimental results when requested.

12. The system of claim 1, wherein said drug discovery experiments are non-animal experiments or animal-based experiments.

13. The system of claim 1, wherein said non-animal experiments are selected from a group consisting of in vitro experiments, in silico experiments, ex vivo experiments, and any combination thereof.

14. The system of claim 1, wherein organisms for said experimental models, are selected from a group consisting prokaryotes, eukaryotes, invertebrates, vertebrates, and any combination thereof.

15. The system of claim 14, wherein said prokaryotes are selected from a group consisting Escherichia coli bacterium, Streptococcus bacterium, archaea and any combination thereof.

16. The system of claim 14, wherein said eukaryotes are selected from a group consisting Saccharomyces cerevisiae, Schizosaccharomyces pombe, Chlamydomonas reinhardtii, Dictyostelium discoideum, and any combination thereof.

17. The system of claim 14, wherein said invertebrates are selected from a group consisting Drosophila melanogaster or Caenorhabditis elegans.

18. The system of claim 14, wherein said vertebrates are selected from a group consisting of rat, mouse, zebra fish, guinea pig, rabbit, pig, hamster and any combination thereof.

19. The system of claim 1, wherein said experimental models are selected from a group consisting in vitro toxicity studies; genetic toxicity studies; DMPK, ADME and PK studies; in vitro efficacy studies; in vitro toxicity studies; ex vivo studies; in vivo efficacy studies; in vivo toxicity studies; and any combination thereof.

20. A method for remote-controlled automated drug testing and screening, comprising steps of:
a. obtaining a system, said system is characterized by:
i. a selection web module for providing selected drug discovery experiments, said selection web module comprises a catalog of available experimental protocols, a list of experimental models, and a list of chemical compounds;
ii. a remote robotic node configured to run said selected drug discovery experiments and retrieve results of these experiments;
iii. a visualization web module configured to analyze and visualize results of said selected drug discovery experiments; and
iv. a machine learning algorithm configured to obtain said experimental results and use said results to recommend experimental parameters in a future experiment; and
b. operating said system.
